(19) United States
(12) Patent Application Publication
(10) Pub. No.: US 2014/0073703 A1
(43) Pub. Date: Mar. 13, 2014

(54) PHYTOL AS A CHOLESTEROL LOWERING AGENT

(71) Applicants: Peter Olofsson, Sjuntorp (SE); Malin Hultqvist, Lund (SE); Rikard Holmdahl, Lund (SE)

(72) Inventors: Peter Olofsson, Sjuntorp (SE); Malin Hultqvist, Lund (SE); Rikard Holmdahl, Lund (SE)

(21) Appl. No.: 13/726,725

(22) Filed: Dec. 26, 2012

Related U.S. Application Data
(62) Division of application No. 12/921,169, filed on Sep. 7, 2010, now abandoned.
(60) Provisional application No. 61/035,167, filed on Mar. 10, 2008.

(51) Int. Cl. A61K 31/045

(52) U.S. Cl. CPC A61K 31/045
USPC 514/739

(57) ABSTRACT

Phytol (3,7,11,15-Tetramethyl-2-hexadecen-1-ol),orderivates thereof, of natural or synthetic origin are used as active ingredient in formulations to lower serum levels of triglycerides and/or cholesterol. Phytol can be administered to patients with disease conditions related to increased levels of cholesterol or triglycerides such as type II diabetes, obesity or other patients in risk of cardiovascular diseases due to elevated cholesterol levels. Phytol can also be administered to healthy individuals to maintain normal levels of serum cholesterol.

### FIELD OF THE INVENTION

The present invention relates to novel methods and compositions for the treatment of dyslipidemia, especially triglyceridemia and hypercholesterolemia in patients with disease conditions related to increased serum levels of cholesterol, especially LDL cholesterol or triglycerides or to maintain normal levels of cholesterol or triglycerides in healthy individuals.

### BACKGROUND OF THE INVENTION

The metabolic syndrome is a cluster of clinical conditions associated with obesity. Abdominal obesity contributes to insulin resistance, a metabolic abnormality linked to the development of type 2 diabetes mellitus and cardiovascular disease (CVD). Approximately 2500 people in the U.S. die from cardiovascular disease (CVD) each day. Each year, CVD claims more lives than the next four leading causes of death combined. Direct and indirect costs of CVD were estimated to be $403 billion in 2006. Insulin resistance generally precedes the development of type 2 diabetes. Currently, an estimated 10 million US adults have diabetes and another 25 million have impaired glucose tolerance (IGT), an intermediate step between insulin resistance and diabetes. The pathophysiologic mechanisms known to increase CVD risk in individuals with insulin resistance include formation of advanced glycation end products, hypertension, proinflammatory and prothrombotic states, and dyslipidemia, including triglyceridemia and hypercholesterolemia, i.e., increased serum levels of triglycerides and cholesterol.

Phytol (CAS nr. 150-86-7) (3,7,11,15-Tetramethyl-2-hexadecen-1-ol) has been reported to be found in various food from fish and meat from ruminant animals and are components of chlorophyll and vitamins E and K. Phytol is extracted from natural chlorophyll sources or chemically derived through series of reactions starting from acetylene and acetone. In the synthetic phytol all isomers are derived while the naturally occurring phytol only consists of the E isomer.

Chlorophyll is present in leaves of green plants to the extent of about 0.2% of wet weight. Phytol is contained in the chlorophyll molecule by ester linkage and represents approximately one-third of the mass of both chlorophylls a and b. Microorganisms, which are present in the rumen of ruminants, are thought to release phytol from chlorophyll, after which phytol is converted into phytanic acid (Patton & Benson, 1966). In fact, 0.1-0.3% of the total lipid content in the lactating cow rumen has been determined as phytol (Patton & Benson, 1966). Because humans are not capable of phytol release from chlorophyll (Baxter, 1968), all phytol and phytanic acid enters the human body via the diet. In particular, ruminant fats, fish and diary products are rich sources of phytol and phytanic acid.

Thus, mammals have been confronted with phytol during all evolution; grass eaters like goats, horses and bovine directly in their food that contain chlorophyll and carnivores by eating herbivores or products like milk and butter from herbivores.

Phytanic acid (3,7,11,15-tetramethyl pentadecen-1-enic acid) is a metabolite of phytol. Due to the presence of the methyl group at the β-carbon of phytanic acid it cannot be degraded by β-oxidation, a major catabolic pathway for fatty acids. It first undergoes α-oxidation to yield pristane acid and CO₂, which in turn is degraded by β-oxidation to yield pristanic acid (Pahan et al., 1994).

Heredopathia atactica polyneuritiformis (Refsum's disease) is a hereditary recessive disorder affecting the nervous system function and characterised by retinitis pigmentosa (RP), hypertrophic peripheral neuropathy and cerebellar ataxia (Refsum, 1976). These patients have an enzymatic defect in the process of alpha-oxidation of fatty acids and therefore lack the function to metabolize phytanic acid which results in accumulation of phytanic acid (Steinberg et al., 1965) (Kahlke & Wagener, 1966). The disease is correlated to the concentration of phytanic acid in the serum.

Administration and kinetic studies of radiolabelled phytol have been performed in patients suffering from Refsum's disease (Kahlke & Wagener, 1966). 72 mg tritium labelled phytol was orally administered to one patient after overnight fasting. Plasma cholesterol esters and triglycerides were determined to be the principal carriers of phytol.

In healthy individuals serum levels of phytanic acid is present at micromolar concentrations (<10 and >3 μM, respectively) (ten Brink et al., 1992). In patients with Refsum's disease the plasma concentration of phytanic acid and pristanic acid can rise to 1300 and 80 μM, respectively (Verhoeven et al., 1998).

Phytanic acid, but not phytol, has been shown to be an RXR (retinoid X receptor) activator (Kitareewan et al., 1996). Phytanic acid has further been postulated to activate both PPARα and RXR in vitro. It was shown in 3T3-L1 cells that phytanic acid activates AP2 mRNA, which mimics the effect of synthetic RXR agonists but not PPARα agonists. This was said to suggest a potentially use of phytanic acid for the treatment of type 2 diabetes and obesity (Schluter et al., 2002). The effect of phytol was not investigated.

Enhanced supply of phytol in the diet of adult mice has been shown to cause proliferation of hepatic peroxisomes. Serum triglyceride levels were decreased after three weeks of phytol feeding, while serum cholesterol levels remained unaffected (Van den Branden et al., 1986).

In EP 1177789 it was demonstrated that phytanic acid can increase and stimulate the transcription of the genes for glucose transporters and glucokinase resulting in increased uptake of glucose in hepatocytes. However, the effect of phytol was not investigated. Phytanic acid was suggested as treatment for non-insulin dependent diabetes and related diseases associated with impaired glucose tolerance.

Phytol and phytanic acid was shown in a mouse teratogenesis models to be able to prevent vitamin A (retinol) induced teratogenic effects (Arnhold et al., 2002).

### BRIEF DESCRIPTION OF THE INVENTION

Provided herein are methods and materials related to preventing and treating disease conditions such as cardiovascular disease, obesity, hypercholesterolemia and type II diabetes. Also provided is the administration of active ingredients to maintain normal healthy levels of cholesterol and triglycerides. For example, the present invention provides methods and materials involved in treating, preventing, ameliorating one or more symptoms associated with, and/or delaying the onset of diseases related to dyslipidemia, especially triglyceridemia and hyperlipidemia, i.e., increased serum levels of triglycerides and cholesterol.

The inventions described herein are based on the demonstration that serum levels of cholesterol, especially LDL (low density lipoprotein) cholesterol, and triglycerides can be lowered by administration of phytol.

Accordingly, the present invention provides methods for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases, comprising the administration to a subject in need thereof a pharmaceutical composition comprising a therapeutically effective amount of phytol and/or phytol derivatives.

Preferably, the present invention provides methods for the prophylaxis, prevention and treatment of hypercholesterolemia, especially prophylaxis, prevention and/or treatment of subjects with elevated serum levels of LDL cholesterol. By elevated serum levels of LDL cholesterol is meant serum levels of above about 2 mmol/L, such as above 3 mmol/L, or above 4 mmol/l, or above 5 mmol/L.

The present invention further provides methods for lowering, controlling and/or maintaining normal levels of serum cholesterol, especially LDL cholesterol and/or triglycerides, comprising the administration to a subject in need thereof a pharmaceutical composition comprising a therapeutically effective amount of phytol and/or phytol derivatives.

The present invention further provides pharmaceutical compositions comprising phytol and/or phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

Preferably, the present invention provides pharmaceutical compositions for the prophylaxis, prevention and treatment of hypercholesterolemia, especially prophylaxis, prevention and/or treatment of subjects with elevated serum levels of LDL cholesterol.

The present invention further provides pharmaceutical compositions comprising phytol and/or phytol derivatives for lowering, controlling and/or maintaining normal levels of serum cholesterol, especially LDL cholesterol, and/or triglycerides.

The invention further provides use of phytol and/or phytol derivatives in the manufacture of a pharmaceutical composition for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

Preferably, the invention further provides use of phytol and/or phytol derivatives in the manufacture of a pharmaceutical composition for the prophylaxis, prevention and/or treatment of hypercholesterolemia, especially in the manufacture of a pharmaceutical composition for the prophylaxis, prevention and/or treatment of subjects with elevated serum levels of LDL cholesterol.

The invention further provides for the use of phytol and/or phytol derivatives in the manufacture of a pharmaceutical composition for lowering, controlling and/or maintaining normal levels of serum cholesterol, especially LDL cholesterol, and/or triglycerides.

The present invention further provides nutritional supplements comprising phytol and/or phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

Preferably, the invention provides nutritional supplements comprising phytol and/or phytol derivatives for the prophylaxis, prevention and/or treatment of hypercholesterolemia, especially nutritional supplements for the prophylaxis, prevention and/or treatment of subjects with elevated serum levels of LDL cholesterol.

The present invention further provides nutritional supplements comprising phytol and/or phytol derivatives for lowering, controlling and/or maintaining normal levels of serum cholesterol, especially LDL cholesterol, and/or triglycerides.

The present invention further provides functional food compositions comprising phytol and/or phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

Preferably, the invention provides functional food comprising phytol and/or phytol derivatives for the prophylaxis, prevention and/or treatment of hypercholesterolemia, especially functional food for the prophylaxis, prevention and/or treatment of subjects with elevated serum levels of LDL cholesterol.

The present invention further provides functional food compositions comprising phytol and/or phytol derivatives for lowering, controlling and/or maintaining normal levels of serum cholesterol, especially LDL cholesterol, and/or triglycerides.

The present invention further provides dietary supplements comprising phytol and/or phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

Preferably, the invention provides dietary supplements comprising phytol and/or phytol derivatives for the prophylaxis, prevention and/or treatment of hypercholesterolemia, especially dietary supplements for the prophylaxis, prevention and/or treatment of subjects with elevated serum levels of LDL cholesterol.

The present invention further provides dietary supplements comprising phytol and/or phytol derivatives for lowering, controlling and/or maintaining normal levels of serum cholesterol, especially LDL cholesterol, and/or triglycerides.

The invention further provides use of phytol and/or phytol derivatives in the manufacture of a nutritional supplement, a functional food or a dietary supplement for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

Preferably, the invention provides use of phytol and/or phytol derivatives in the manufacture of a nutritional supplement, a functional food or a dietary supplement for the prophylaxis, prevention and/or treatment of hypercholesterolemia, especially for the prophylaxis, prevention and/or treatment of subjects with elevated serum levels of LDL cholesterol.

The invention further provides use of phytol and/or phytol derivatives in the manufacture of a nutritional supplement, a functional food or a dietary supplement for lowering, controlling and/or maintaining normal levels of serum cholesterol, especially LDL cholesterol, and/or triglycerides.

Preferably the prophylaxis, prevention and/or treatment of subjects with elevated serum levels of LDL cholesterol according to the invention will lead to selective reduction in serum levels of LDL cholesterol as compared to serum levels of HDL (high density lipoprotein) cholesterol. Preferably, the selective reduction of the serum levels of LDL cholesterol is more than 25%, such as more than 30%, more than 40%, or more than 50%, while the reduction of serum levels of HDL cholesterol is less than 25%, such as less than 20%, or less than 15%, or less than 10%.

Subjects that can be treated with phytol and/or phytol derivatives include humans, pets and husbandry animals.

Preferred phytol derivatives that be used according to the invention include, but not being limited to, phytol esters.

“Phytol esters” that can be used according to the invention includes, but not being limited to, phytyl acetate, phytyl glycerate, phytyl citrate, phytyl succinate, phytyl phosphates, and phytyl esters of fatty acids, such as phytyl palmitate, phytyl oleate, phytyl stearate, phytyl linolate, phytyl linoleate.

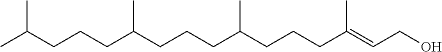 [SMILES: C/C(=C\CO)CCCC(C)CCCC(C)CCCC(C)C]

3,7,11,15-Tetramethyl-hexadec-2-en-1-ol (Phytol);

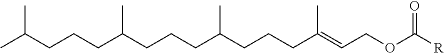

Generic structure of phytol esters, where R can be derived from any carboxylic acid.

Phytol esters and other phytol derivatives are disclosed in WO 2007/104790 and the corresponding U.S. Ser. No. 11/724,354, the content of which hereby is incorporated by reference.

Further preferred phytol derivatives that be used according to the invention includes metabolites of phytol, wherein phytanic acid and phytenic acid and derivatives thereof are preferred. Derivatives of phytanic acid and phytenic acid includes, but not limited to, phytol esters hydroxy-phytanic acid or hydroxy-phytenic acid, especially 2-hydroxy-phytenic acid or 2-hydroxy-phytenic ester, hydroxy-phytanic esters, phytanic amides, phytenic amides, hydroxy-phytanic amides, hydroxy-phytenic amides, hydrocarbon esters, phospholipid esters and triacylglyceryl esters, with long chain n-alkyl esters, preferably C12-C22. Derivatives of phytanic acid and phytenic acid are disclosed in U.S. Pat. No. 6,784,207, the content of which hereby is incorporated by reference.

It is contemplated that any method or composition described herein can be implemented with respect to any other method or composition described herein.

The use of the word “a” or “an” when used in conjunction with the term “comprising” in the claims and/or the specification may mean “one”, but it is also insistent with the meaning of “one or more”, “at least one”, and “one or more than one”.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions, and/or rearrangements may be made within the scope of the invention without departing from the spirit thereof, and the invention includes all such substitutions, modifications, additions and/or rearrangements.

### DESCRIPTION OF DRAWINGS

The following drawings form part of the present specification and are included to further demonstrate certain aspect of the present invention: The invention may be better understood by reference to one or more of these drawings in combination with the detailed description of specific embodiments presented herein.

FIG. 1 is a graph comparing the triglyceride and cholesterol lowering effect of phytol in rats. The rats were treated with oral administration of phytol at 125 mg daily.

FIG. 2 is a graph comparing the triglyceride and cholesterol lowering effect of phytol in pigs. The pigs were treated with oral administration of phytol at 10 g daily.

FIG. 3 is a graph showing the effect of increased dose of phytol in pigs. The pigs were treated with oral administration of 1.25; 2.5; 5 and 10 g phytol daily for two weeks, respectively.

FIG. 4 is a graph comparing the cholesterol lowering effect of phytol in obese ob/ob mice. The mice were treated with oral administration of phytol 0.75% weight evenly distributed in powdered chow.

FIG. 5 is a graph comparing the cholesterol and LDL-Cholesterol lowering effect of phytol in rats. The rats were treated with oral administration of phytol at 1% weight evenly distributed in powdered chow.

FIG. 6 is a graph comparing the cholesterol and LDL-Cholesterol lowering effect of phytol in mice. The mice were treated with oral administration of phytol at 1% weight evenly distributed in powdered chow.

FIG. 7 is a graph comparing the cholesterol and LDL-Cholesterol lowering effect of Phytol in rats. The rats were treated with oral administration of phytol at 400 mg every second day for three weeks.

### DETAILED DESCRIPTION OF THE INVENTION

The present inventors have demonstrated that phytol, of natural or synthetic origin can be used as active ingredient in formulations to lower serum levels of triglycerides and/or cholesterol, especially LDL cholesterol. Phytol and/or derivatives thereof, can be administered to patients with disease conditions related to increased serum levels of cholesterol or triglycerides such as type II diabetes, obesity or other patients in risk of atherosclerosis or other cardiovascular diseases due to elevated serum cholesterol levels. Phytol and/or derivatives thereof, can also be administered to healthy individuals to lower and/or maintain normal serum levels of cholesterol, especially LDL cholesterol and triglycerides as a prophylactic measure. Phytol, and/or derivates thereof, of natural or synthetic origin can be used as active ingredient in formulations to lower serum levels of triglycerides and/or cholesterol, especially LDL cholesterol.

### Formulation and Administration

Phytol and/or phytol esters can be administered orally in various forms such as granules, tablets, pills, capsules, suspensions or liquid. Pharmaceutical grade organic or inorganic carriers, excipients and/or diluents suitable for oral use can be used to make up compositions containing the therapeutically active compounds. Diluents known to the art include aqueous media, vegetable and animal oils and fats. Excipient such as a suitable antioxidant can be used, the antioxidant can be α-tocopherol or ascorbic acid. The pharmaceutical composition will generally contain from 5-100% by weight of active ingredient. The subject composition will generally be administered daily, at a daily dose between at least 100 mg and 100 g, usually between 500 and 5000 mg. The amount will vary with general health of the individual and the response of the individual. The phytol to be used according to the present invention can be of synthetic or natural origin. Natural phytol can be extracted from plants like for example green leaves from plants with high chlorophyll content like mulberry leaves or spinach.

### Pharmacokinetics

Dietary phytol given to animals like (rat, mouse, rabbit and chinchillas) at doses 1-5% in the food leads to accumulation in both liver and serum. At the 1% level no phytol was distributed to the livers. If phytol is subtracted from the food the serum and liver values of accumulated phytol drops rapidly (Steinberg et al., 1966).

The rat has a capacity to absorb as much as 0.2 g phytol/kg bodyweight in to the intestinal lymphatic after oral administration (Baxter & Steinberg, 1967; Baxter et al., 1967). This transportation takes place within 20 hours after administration. More than 50% of orally administered phytol is absorbed of which more than 70% is absorbed via the intestinal lymphatics. If the phytol is mixed with bile salts prior to administration the absorption increases. Orally administered phytol to rats is well absorbed (30-66% of administered dose). In the case when rats were fed a containing 5% phytol it was shown that the rats had the capacity to rapidly absorb and degrade phytol (Mize et al., 1966).

The absorption of phytol given as chlorophyll is much lower compared to when given as pure phytol. For example spinach can contain as much as 1-2% phytol. But when administered orally to rat only about 1-2% of the phytol content is absorbed. Hence, when phytol is bound as chlorophyll it is to most extent excreted with the faeces.

### Toxicity

Both the acute oral LD50 in rats and acute dermal LD50 in rabbits exceed 5 g/kg. No evidence of growth inhibition or toxicity was observed in rats fed 0.5% phytol in the diet for 15 months (Steinberg et al., 1966). Subcutaneous irritation test on humans at 10% concentration produced no irritation (Kligman, 1966), (Kligman & Epstein, 1975)

### EXAMPLES

The following examples are included to demonstrate preferred embodiments of the invention. It should be appreciated by those of skill in the art that the techniques disclosed in the examples which follows represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit ns scope of the invention.

### Example 1

### Phytol Treatment Healthy Rats

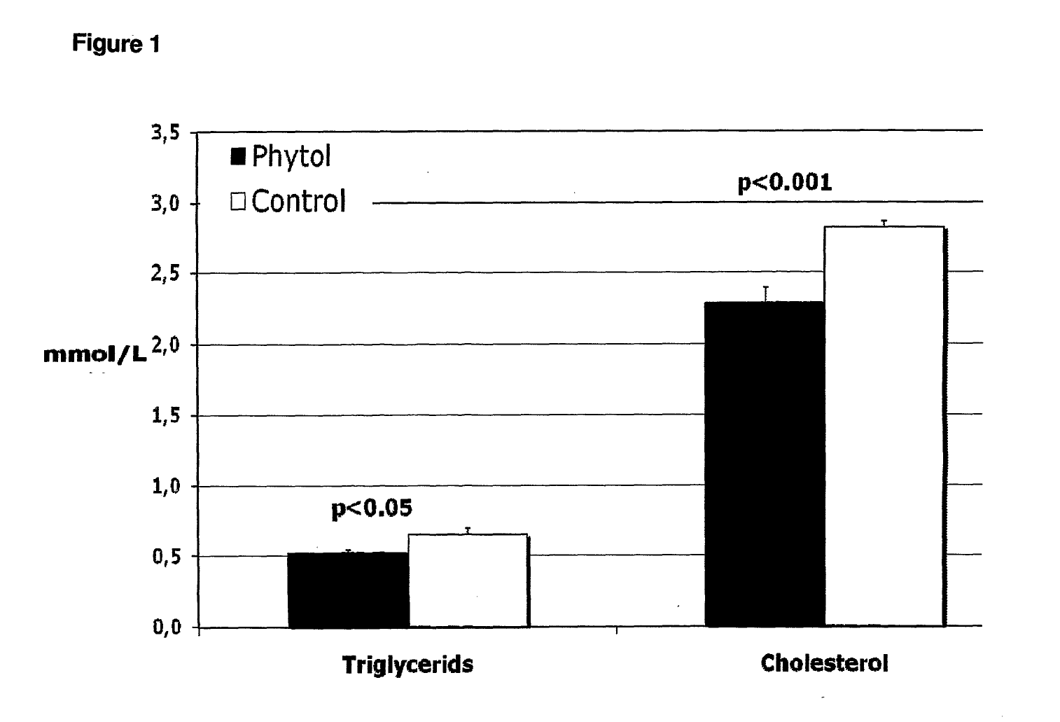

Triglyceride and cholesterol lowering effect after oral administration of phytol was validated in serum from healthy rats treated with phytol. Phytol (FIG. 1; black bars) was administrated to rats at 1 weight percentage phytol mixed in the food. Analysis was made in comparison with control (FIG. 1; white bars) untreated rats. After 4 days of treatment the serum levels of triglycerides (p<0.05) and cholesterol (p<0.001) were significantly reduced in the rats given phytol.

### Example 2

### Phytol Treatment Healthy Pigs

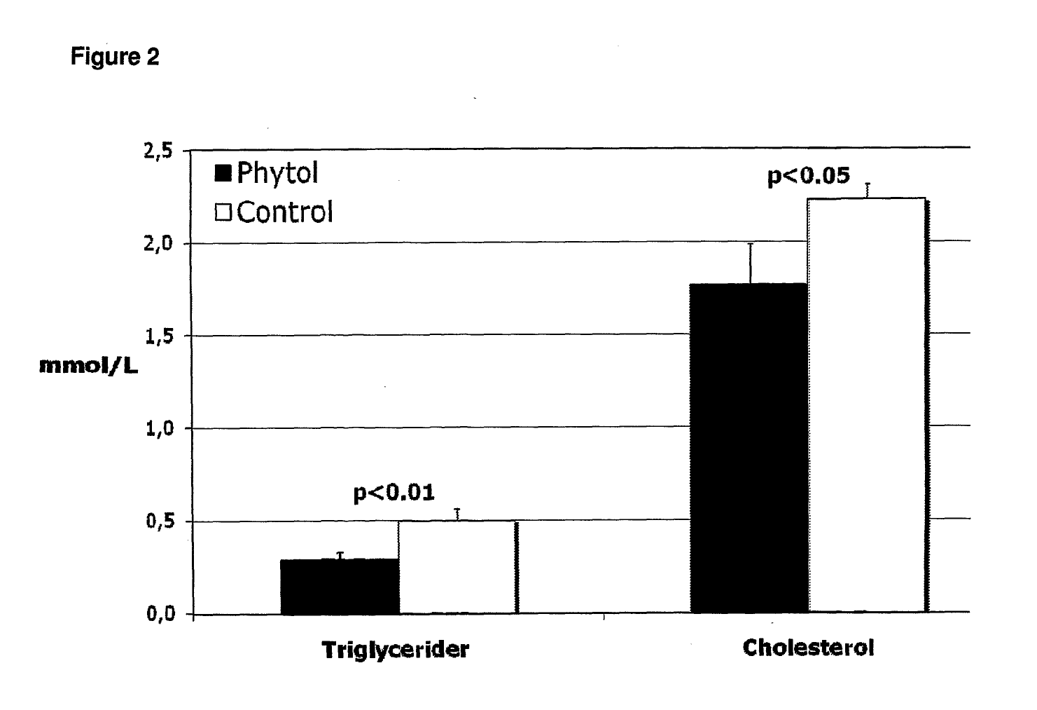

Triglyceride and cholesterol lowering effect after oral administration of phytol was validated in serum from healthy pigs treated with phytol. Phytol (FIG. 2.; black bars) was administrated to pigs at 10 g phytol daily mixed in the food. Analysis was made in comparison with control (FIG. 2.; white bars) untreated pigs. After 1 wk of treatment the serum levels of triglycerides (p<0.01) and cholesterol (p<0.05) were significantly reduced in the pigs given phytol.

### Example 3

### Phytol Treatment Healthy Pigs

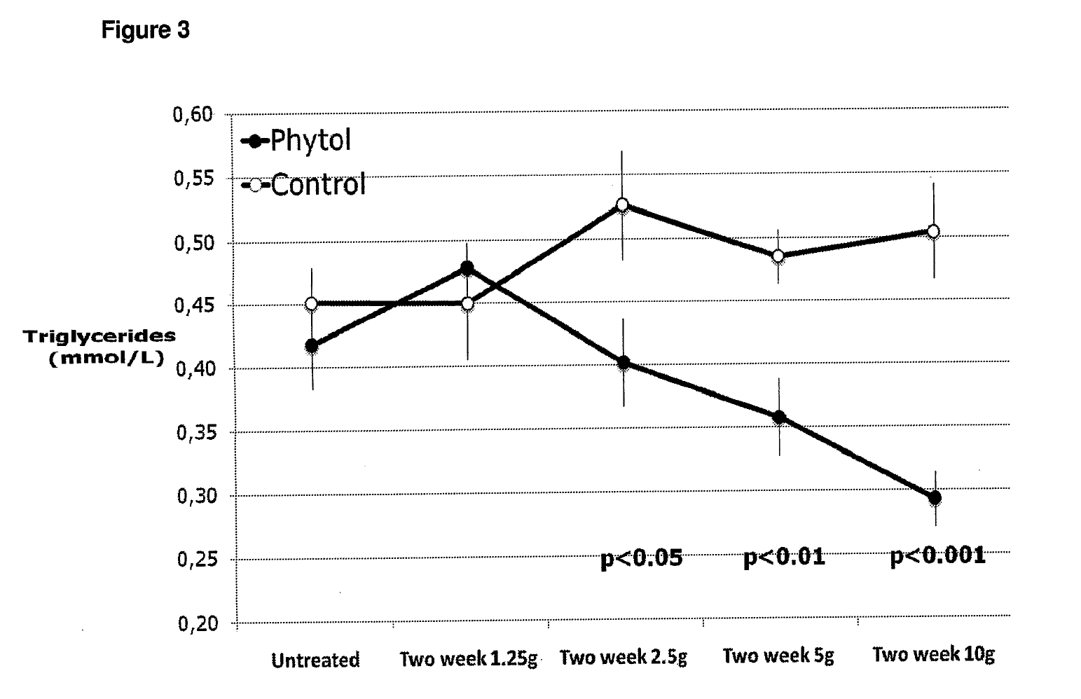

Triglyceride lowering effect after oral administration of phytol was validated in serum from healthy pigs treated with phytol. Phytol (FIG. 3.; black circles) was administrated to pigs at 1.25, 2.5, 5 and 10 g phytol daily. Analysis was made in comparison with control (FIG. 3.; open circles) pigs given corn oil as control. Serum levels of triglycerides were significantly reduced in the pigs given phytol in a dose dependent manner (with 2.5 g phytol per day p<0.05, with 5 g phytol per day p<0.01 and with 10 g phytol per day p<0.001).

### Example 4

### Phytol Treatment obese (ob/ob) mice

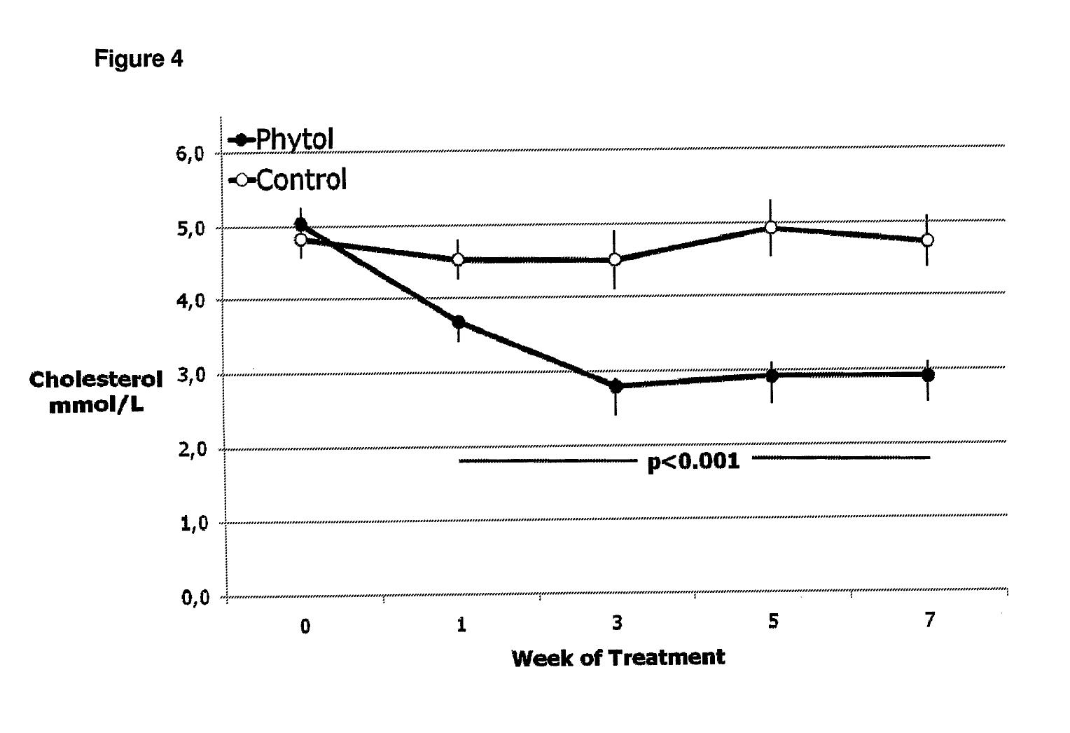

Cholesterol lowering effect after oral administration of phytol was validated in serum from obese (ob/ob) mice treated with phytol. Phytol (FIG. 4; black circles) was administrated to the mice at 0.75 weight percentage phytol mixed in the food. Analysis was made in comparison with control (FIG. 4; open circles) untreated mice. During 5 weeks of treatment the serum levels of Cholesterol (p<0.001) were significantly reduced in the mice given phytol.

### Example 5

### Phytol Treatment Healthy Rats

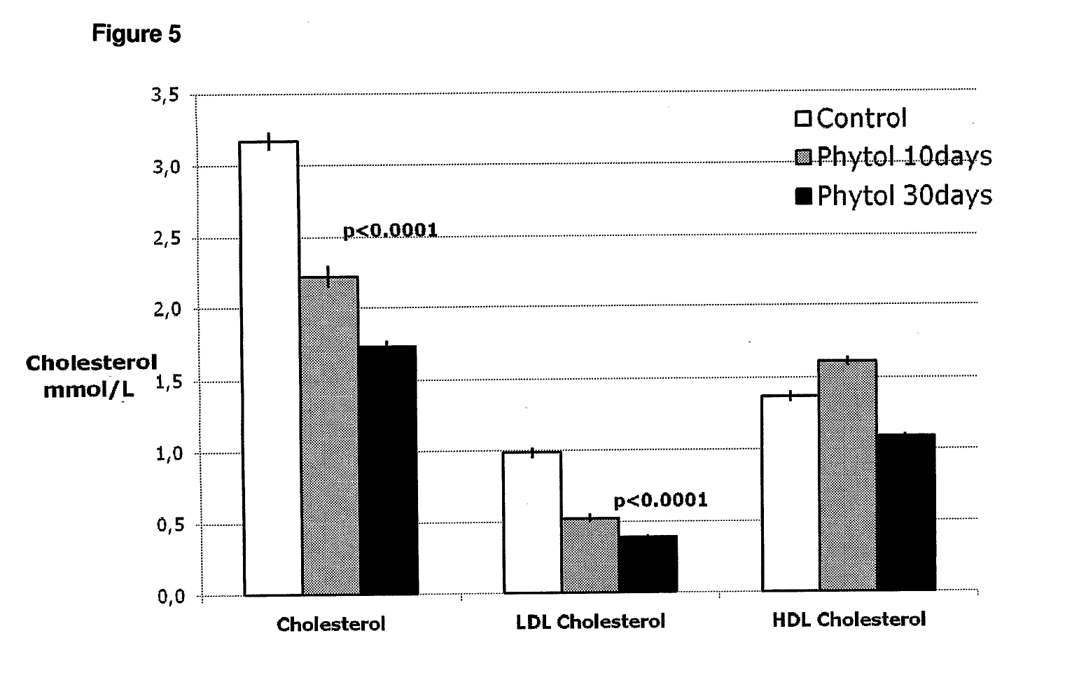

Cholesterol and LDL-Cholesterol lowering effect after oral administration of phytol was validated in serum from healthy rats treated with phytol. Phytol (FIG. 5; black bars) was administrated to rats at 1 weight percentage phytol mixed in the food for thirty days. Analysis was made in comparison with control (FIG. 5; white bars) untreated rats. After ten days of treatment the serum levels of serum cholesterol (p<0.0001) and LDL-cholesterol (p<0.0001) were significantly reduced in the rats given phytol. After thirty days of treatment the serum levels of serum cholesterol and LDL-cholesterol were even more reduced in the rats given phytol

### Example 6

### Phytol Treatment Healthy mice

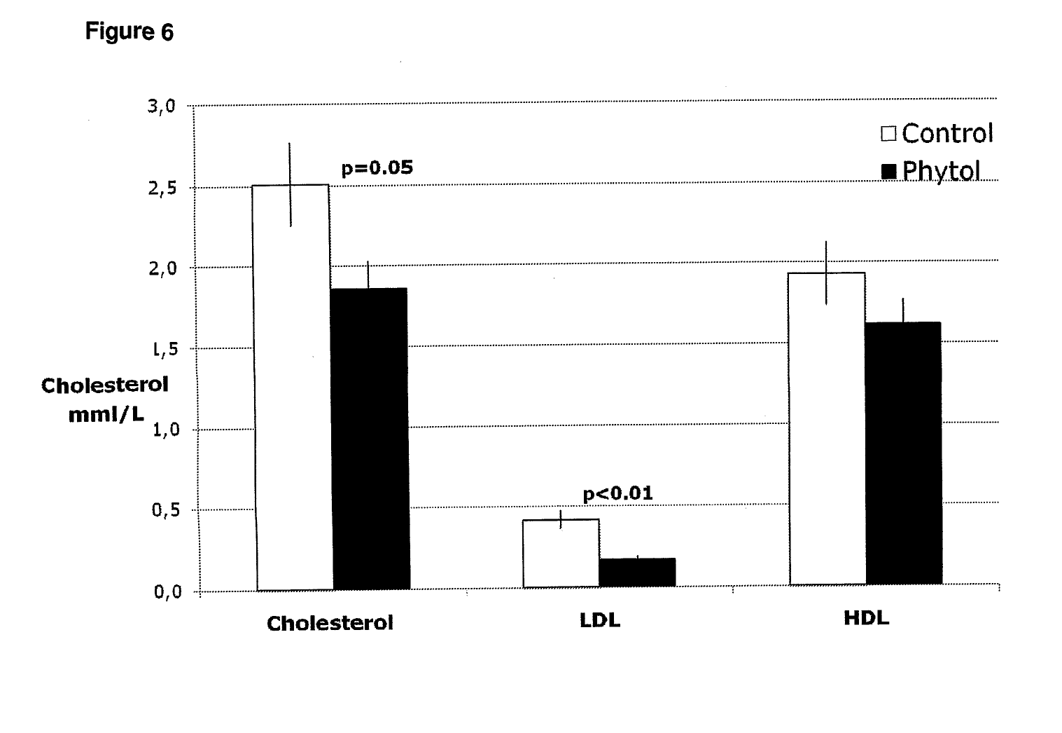

Cholesterol and LDL-Cholesterol lowering effect after oral administration of phytol was validated in serum from healthy mice treated with phytol. Phytol (FIG. 6; black bars) was administrated to mice at 1 weight percentage phytol mixed in the food for thirty days. Analysis was made in comparison with control (FIG. 6; white bars) untreated mice. After thirty days treatment the serum levels of serum cholesterol (p=0.05) and LDL-cholesterol (p<0.01) were significantly reduced in the mice given phytol.

### Example 7

### Phytol Treatment Healthy Rats

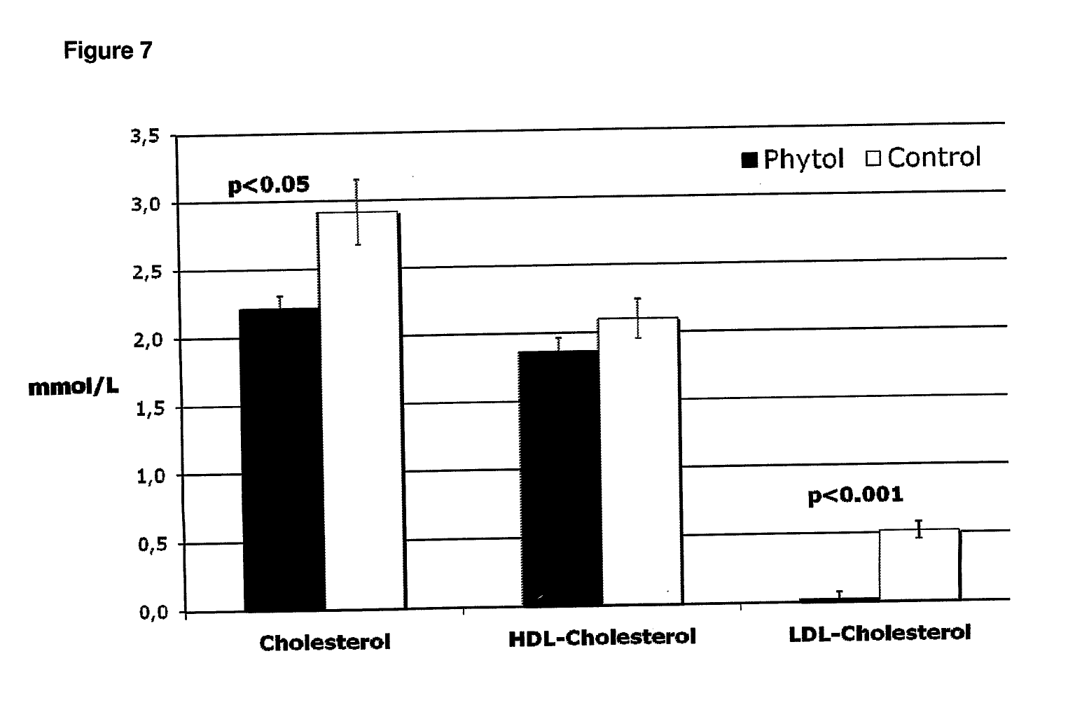

Cholesterol and LDL-Cholesterol lowering effect after oral administration of phytol was validated in serum from healthy rats treated with phytol. Phytol (FIG. 7; black bars) was administrated to rats at 400 mg phytol administered every second day for three weeks. Analysis was made in comparison with control (FIG. 7; white bars) untreated rats. After three weeks treatment the serum levels of serum cholesterol (p<0.05) and LDL-cholesterol (p<0.001) were significantly reduced in the rats given phytol.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention: More specifically, it will be apparent that certain agents which are both chemically and physiological related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arnhold T., Elmazar M. M., and Nau H. (2002). Prevention of vitamin A teratogenesis by phytol or phytanic acid results from reduced metabolism of retinol to the teratogenic metabolite, all-trans-retinoic acid. Toxicol Sci 66: 274-82.
Baxter J. H. (1968). Absorption of chlorophyll phytol in normal man and in patients with Refsum's disease. J Lipid Res 9: 636-41.
Baxter J. H., and Steinberg D. (1967). Absorption of phytol from dietary chlorophyll in the rat. J Lipid Res 8: 615-20.
Baxter J. H., Steinberg D., Mize C. E., and Avigan J. (1967). Absorption and metabolism of uniformly 14C-labeled phytol and phytanic acid by the intestine of the rat studied with thoracic duct cannulation. Biochim Biophys Acta 137: 277-90.
Kahlke W., and Wagener H. (1966). Conversion of H3-phytol to phytanic acid and its incorporation into plasma lipid fractions in heredopathia atactica polyneuritiformis. Metabolism 15: 687-93.
Kitareewan S., Burka L. T., Tomer K. B., Parker C. E., Deterding L. J., Stevens R. D., Forman B. M., Mais D. E., Heyman R. A., McMorris T., and Weinberger C. (1996). Phytol metabolites are circulating dietary factors that activate the nuclear receptor RXR. Mol Biol Cell 7: 1153-66.
Kligman A. M. (1966). The identification of contact allergens by human assay. 3. The maximization test: a procedure for screening and rating contact sensitizers. J Invest Dermatol 47: 393-409.
Kligman A. M., and Epstein W. (1975). Updating the maximization test for identifying contact allergens. Contact Dermatitis 1: 231-9.
Mize C. E., Avigan J., Baxter J. H., Fales H. M., and Steinberg D. (1966). Metabolism of phytol-U-14C and phytanic acid-U-14C in the rat. J Lipid Res 7: 692-7.
Pahan K., Gulati S., and Singh I. (1994). Phytanic acid alpha-oxidation in rat liver mitochondria. Biochim Biophys Acta 1201: 491-7.
Patton S., and Benson A. A. (1966). Phytol metabolism in the bovine. Biochim Biophys Acta 125: 22-32.
Refsum S. (1976). Heredopathia atactica polyneurotiformis: therapeutic and pathogenetic aspects. Riv Patol Nery Ment 97: 115-26.
Schluter A., Yubero P., Iglesias R., Giralt M., and Villarroya F. (2002). The chlorophyll-derived metabolite phytanic acid induces white adipocyte differentiation. Int J Obes Relat Metab Disord 26: 1277-80.
Steinberg D., Avigan J., Mize C., and Baxter J. (1965). Phytanic Acid Formation and Accumulation in Phytol-Fed Rats. Biochem Biophys Res Commun 19: 412-6.
Steinberg D., Avigan J., Mize C. E., Baxter J. H., Cammermeyer J., Fales H. M., and Highet
P. F. (1966). Effects of dietary phytol and phytanic acid in animals. J Lipid Res 7: 684-91.
ten Brink H. J., Schor D. S., Kok R. M., Poll-The B. T., Wanders R. J., and Jakobs C. (1992). Phytanic acid alpha-oxidation: accumulation of 2-hydroxyphytanic acid and absence of 2-oxophytanic acid in plasma from patients with peroxisomal disorders. J Lipid Res 33: 1449-57.
Van den Branden C., Vamecq J., Wybo I., and Roels F. (1986). Phytol and peroxisome proliferation. Pediatr Res 20: 411-5.
Verhoeven N. M., Wanders R. J., Poll-The B. T., Saudubray J. M., and Jakobs C. (1998). The metabolism of phytanic acid and pristanic acid in man: a review. J Inherit Metab Dis 21: 697-728.

1. A method for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases, comprising the administration to a subject in need thereof a pharmaceutical composition comprising a therapeutically and/or therapeutically effective amount of phytol and/or one or more phytol derivatives.

2. The method according to claim 1, for the prophylaxis, prevention and/or treatment of hypercholesterolemia.

3. The method according to claim 2, for the prophylaxis, prevention and/or treatment of elevated serum levels of LDL cholesterol.

4. The method according to any of claims 1 to 3, wherein the composition comprises phytol.

5. A method for lowering, controlling and/or maintaining normal levels of serum cholesterol and/or triglycerides, comprising the administration to a subject in need thereof a pharmaceutical composition comprising a therapeutically effective amount of phytol and/or one or more phytol derivatives.

6. The method according to claim 5, for lowering, controlling and/or maintaining normal levels of serum cholesterol.

7. The method according to claim 6, wherein the cholesterol is LDL cholesterol.

8. The method according to any of claims 5 to 7, wherein the composition comprises phytol.

9. A pharmaceutical composition comprising phytol and/or one or more phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

10. The pharmaceutical composition according to claim 9 for the prophylaxis, prevention and/or treatment of hypercholesterolemia.

11. The pharmaceutical composition according to claim 10, for the prophylaxis, prevention and/or treatment of elevated serum levels of LDL cholesterol.

12. The pharmaceutical composition according to any of claims 9 to 11, wherein the composition comprises phytol.

13. A pharmaceutical composition comprising phytol and/or one or more phytol derivatives for lowering, controlling and/or maintaining normal levels of serum cholesterol and/or triglycerides.

14. The pharmaceutical composition according to claim 13 for lowering, controlling and/or maintaining normal levels of serum cholesterol.

15. The pharmaceutical composition according to claim 14, wherein the cholesterol is LDL cholesterol.

16. The pharmaceutical composition according to any of claims 13 to 15, wherein the composition comprises phytol.

17. A nutritional supplement comprising phytol and/or a one or more phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

18. A nutritional supplement comprising phytol and/or one or more phytol derivatives for lowering, controlling and/or maintaining normal levels of serum cholesterol and/or triglycerides.

19. The nutritional supplement according to claim 18, wherein the cholesterol is LDL cholesterol.

20. A functional food composition comprising phytol and/or one or more phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

21. A functional food composition comprising phytol and/or one or more phytol derivatives for lowering, controlling and/or maintaining normal levels of serum cholesterol and/or triglycerides.

22. The composition according to claim 21, wherein the cholesterol is LDL cholesterol.

23. A dietary supplement comprising phytol and/or one or more phytol derivatives for the prophylaxis, prevention and/or treatment of hypercholesterolemia, triglyceridemia and/or hyperlipidemia, as well as related conditions such as obesity, insulin resistance, type II diabetes, atherosclerosis and related cardiovascular diseases.

24. A dietary supplement comprising phytol and/or one or more phytol derivatives for lowering, controlling and/or maintaining normal levels of serum cholesterol and/or triglycerides.

25. The dietary supplement according to claim 24, wherein the cholesterol is LDL cholesterol.
